(19) United States
(12) Patent Application Publication
(10) Pub. No.: US 2021/0299292 A1
(43) Pub. Date: Sep. 30, 2021

(54) Method to Kill Respiratory Viruses and Microbes by Water Mist Charge

(71) Applicants: Kenneth Tsan He, (US); Zongyan Tsan He, (US)

(72) Inventors: Helena Ai He, Kendall Park, NJ (US); Kenneth Tsan He, Kendall Park, NJ (US)

(21) Appl. No.: 16/833,640

(22) Filed: Mar. 29, 2020

(51) Int. Cl. A61L 2/00; A61L 2/22; A61L 2/03; A61L 2/26

(52) U.S. Cl. CPC A61L 2/0088; A61L 2/22; A61L 2202/15; A61L 2/26; A61L 2/03

(57) ABSTRACT

The invention relates to an electrostatic aerosol disinfection and sterilization method. The method includes the following steps: atomizing liquid water without any medicine to obtain atomized particles of liquid water; and carrying the atomized particles on Static electricity of a first polarity; transporting atomized particles with static electricity to a predetermined surface of an object to be disinfected, the objects to be disinfected have static electricity of a first polarity; when the atomized particles reach the object to be disinfected When the predetermined surface is changed, the electrostatic polarity of the object to be disinfected is changed, so that the object to be disinfected has static electricity of a second polarity, wherein the second polarity is opposite to the polarity of the first polarity. The present invention is capable of treating and slowing down various respiratory diseases caused by viruses, including new pneumonia that threatens people all over the world, greatly improves the effect of disinfection and sterilization, is not only safe and reliable, but also has simple operation and low cost.

### BACKGROUND

At present, pneumonia caused by the new coronavirus is spreading all over the world. So far, there are no methods and drugs capable of killing viruses that remain in the respiratory tract.

As we all know, physical disinfection, such as chemical disinfectants, boiling, Pasteur heating, ultraviolet radiation, steam, dry heat, and radiation, are mainly used in medicine for disinfection and sterilization. However, the above methods are more harmful to the human body and are more commonly used in clinical and daily life Chemicals treat the surface of the skin, wounds, mouth, nasopharynx, eyes, and ears, but all kinds of chemicals have their limitations, such as irritating the human body, causing side effects, or poor chemical stability, Volatile deterioration.

In recent years, cold plasma has been used more and more in the field of biomedicine, especially when cold plasma is close to or slightly above room temperature, it will not cause obvious thermal damage to human body and biological tissues, does not pollute the surrounding environment, and cause harm to medical devices. It will not cause thermal deformation and damage, and can effectively inactivate various pathogenic microorganisms such as bacteria, fungi and viruses, which makes up for the shortcomings of high-pressure steam, chemical and radiation methods. At present, cold plasma has achieved very good research results in sterilization and disinfection, material surface modification, dental treatment, beauty, hemostasis and anti-inflammatory, wound healing, skin disease treatment, and tumor treatment. Combining plasma with the field of clinical medicine can be used as an adjunct to surgery, or to assist drugs to prevent and treat diseases.

In the prior art, there have been related sterilization technologies using plasma. For example, Chinese patent 200910164942.2 discloses a method of using water mist particles to deliver drugs or ozone to a diseased site for sterilization. However, according to the experiments of the present inventors, it is difficult for these atomized particles carrying drugs or ozone to accumulate and settle on the diseased area to form a drug solution or ozone water, so the effect of disinfection and sterilization is not good, which affects the effect of patient treatment and increases Cost of treatment.

### SUMMARY OF THE INVENTION

Based on this, it is necessary to provide an electrostatic aerosol disinfection method and equipment for the above problems, which can effectively kill viruses that remain in the respiratory tract and greatly improve the effect of disinfection and sterilization. It is not only safe and reliable, but also simple to operate ,low cost.

The invention provides an electrostatic aerosol disinfection and sterilization method, which comprises the following steps: atomizing liquid water containing no medicine to obtain atomized particles of liquid water; and carrying the electrostatic charges of the first polarity on the atomized particles; Transporting the atomized particles with static electricity to a predetermined surface of the object to be disinfected, the object to be disinfected carries static electricity of a first polarity; when the atomized particles reach the predetermined surface of the object to be disinfected, changing the electrostatic polarity of the object to be disinfected is such that the object to be disinfected has static electricity of a second polarity, wherein the second polarity is opposite to the polarity of the first polarity.

Further, in the electrostatic aerosol disinfection and sterilization method described above, the liquid water is sterilized in advance.

Further, in the aforementioned electrostatic aerosol sterilization method, the electrostatic voltage between the first electrostatic polarity and the second electrostatic polarity is not lower than 300 volts.

Further, in the above-mentioned electrostatic aerosol sterilization and sterilization method, the step of atomizing liquid water containing no medicine to obtain atomized particles of liquid water specifically includes: atomizing liquid water by pressure spraying to obtain liquid water Atomized particles.

Further, in the above-mentioned electrostatic aerosol disinfection and sterilization method, the step of bringing the atomized particles with a static electricity of a first polarity specifically includes directly outputting the liquid water with a high voltage having a first polarity. The electrodes are connected so that the atomized particles have static electricity of a first polarity.

Further, in the above-mentioned electrostatic aerosol disinfection and sterilization method, the step of bringing the atomized particles with a first-polar static electricity specifically includes: passing the liquid water under pressure through a metal connected to a high-voltage electrode. The nozzle sprays atomized particles with static electricity, and the high-voltage electrode has a first polarity.

Further, in the above-mentioned electrostatic aerosol disinfection and sterilization method, the step that the object to be disinfected has static electricity of a first polarity includes:

Keep the human body insulated from the ground or other electrical conductors, so that the human body directly or indirectly contacts the high-voltage electrode with the first polarity.

Further, in the aforementioned electrostatic aerosol sterilization method, the method further includes:

Calculate the time that the atomized particles reach the predetermined surface of the object to be disinfected, and determine whether the atomized particles reach the predetermined surface of the object to be disinfected according to the time.

Further, in the above-mentioned electrostatic aerosol disinfection and sterilization method, the step of calculating the time when the atomized particles reach a predetermined surface of the object to be disinfected specifically includes:

Calculating the distance required for the atomized particles to reach a predetermined surface of the object to be disinfected;

Measuring the initial speed of the atomized particle airflow, and calculating the flow rate of the atomized particle airflow according to the initial speed;

Estimating the resistance of the atomized particle airflow to a predetermined surface process of the object to be disinfected;

A function model is established according to the flow, resistance and distance of the atomized particle airflow, and the time required for the atomized particles to reach a predetermined surface of the object to be disinfected is calculated through the function model.

In addition, the present invention also provides an electrostatic aerosol disinfection and sterilization system, including:

Atomization module, for atomizing liquid water without any medicine to obtain atomized particles of liquid water;

An electrification module, configured to bring the atomized particles and the object to be disinfected with static electricity of a first polarity;

A conveying module for conveying electrostatically atomized particles to a predetermined surface of an object to be disinfected;

A polarity switching module, configured to change the electrostatic polarity of the object to be disinfected when the atomized particles reach a predetermined surface of the object to be disinfected, so that the object to be disinfected has static electricity of a second polarity, The second polarity is opposite to the first polarity.

Further, in the above-mentioned electrostatic aerosol disinfection and sterilization system, the electrostatic aerosol disinfection and sterilization system further includes:

A calculation module, configured to calculate a time when the atomized particles reach a predetermined surface of an object to be disinfected;

And a determining module, configured to determine whether the atomized particles reach a predetermined surface of the object to be disinfected according to the time.

In addition, the present invention also provides an electrostatic aerosol disinfection and sterilization device, which includes the electrostatic aerosol disinfection and sterilization system as described above.

The electrostatic aerosol disinfection method, system and equipment of the present invention calculate the time when the atomized particles reach a predetermined surface of the object to be disinfected, control the electrostatic polarity of the object to be disinfected according to the time, and The electrostatically atomized particles will generate active particles such as plasma and ozone when the predetermined surface of the disinfected object is discharged and settled, which effectively inhibits the propagation of pathogenic microorganisms and greatly improves the effect of disinfection and sterilization. It is not only safe and reliable, but also simple to operate ,low cost.

### BRIEF DESCRIPTION OF THE DRAWINGS

FIG. 1 is a schematic diagram of a specific process of an electrostatic aerosol disinfection method according to the present invention.

FIG. 2 is a specific schematic diagram for calculating the time when the atomized particles reach a predetermined surface of the object to be disinfected in FIG. 1.

FIG. 3 is a schematic diagram of the method of the present invention for killing nasal viruses.

FIGS. 4 and FIG. 5 show the comparison of the number of colonies after using the system to spray ordinary water mist and charged water mist to volunteers' nasal cavity.

### DETAILED DESCRIPTION

The invention provides an electrostatic aerosol disinfection and sterilization method, which includes the following steps: atomizing liquid water without any medicine to obtain atomized particles; bringing the atomized particles to a first-polar static electricity; The electrostatically atomized particles are delivered to a predetermined surface of the object to be disinfected, and the object to be disinfected has static electricity of a first polarity; when the atomized particles reach the predetermined surface of the object to be disinfected, the to-be-sterilized particle is changed The electrostatic polarity of the object causes the object to be sterilized to have a static electricity of a second polarity, wherein the second polarity is opposite to the polarity of the first polarity. In the present invention, the atomized particles atomized with liquid water first have the same polarity of static electricity as the object to be disinfected, so that when the atomized particles reach a predetermined surface of the object to be disinfected, the object to be disinfected is made With the opposite polarity of static electricity, due to the attractive force of the opposite charge, the atomized particles quickly settle on the surface of the object and lose its charge. The plasma, ozone, chloride ions, salt ions generated in the process, Under the multiple effects of metal ions and pulse discharge, the reproduction of pathogenic microorganisms on the object to be disinfected is effectively suppressed or killed, which greatly improves the effect of disinfection and sterilization.

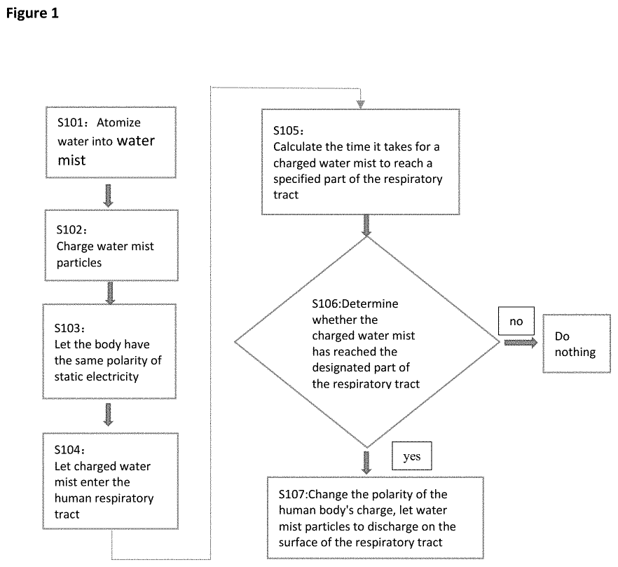

Please refer to FIG. 1. It is a schematic diagram of a specific process of the electrostatic aerosol disinfection and sterilization method of the present invention. The method specifically includes:

Step S101: Atomizing Liquid Water Without any Medicine to Obtain Atomized Particles of Liquid Water;

In specific implementation, the present invention uses non-medicinal liquid water for disinfection, and the liquid water may be clean water; the present invention may also use a certain concentration of saline to further increase the effect of disinfection and sterilization.

The present invention adopts a wide range of clean water or salt water and other raw materials with low price as the liquid water for generating aerosol. Under conditions, the liquid water itself must be sterilized beforehand and must not contain pathogenic microorganisms. In order to prevent the ultrasonic vibrator from scaling, the electrolyte concentration of liquid water should not be too high. If it is necessary to use salt water to strengthen the disinfection and sterilization effect, anti-corrosion ceramic film vibration plate should be used. And regular cleaning. The concentration of brine is preferably lower than the saturated concentration. In this embodiment, the liquid water is clean water or brine.

In this step, the liquid water can be atomized in various ways. In this embodiment, ultrasonic atomization is preferred. Ultrasonic atomization is a high-frequency resonance generated by a ceramic sheet with high-frequency oscillation (frequency is generally 1.7 MHz to 2.4 MHz). , The liquid water molecular structure is broken up to produce water mist. Compared with heating atomization, ultrasonic atomization can save 90% of energy. Negative ions are also released during the atomization process, which generates an electrostatic reaction with the smoke and dust floating in the air to cause precipitation, and at the same time, it can remove some harmful substances such as formaldehyde and bacteria to purify the air. But this purification ability is weak. The use of high vibration intensity ultrasound and low ventilation levels can effectively atomize the liquid and deliver it to various parts of the body, including the nasopharynx, mouth, eyes, ears, and pelvis. However, due to the cavitation effect of ultrasound, the drugs in the liquid are easily broken down and destroyed. Therefore, high-concentration, high-viscosity drug solutions are not suitable.

Another advantage of ultrasonic atomization is that the atomization efficiency is high, the particle size of the atomized particles is small (theoretically, it can be controlled at 1-5 microns), and it is uniform and stable. But because of this, ultrasonic atomized particles are not easy to aggregate and settle on solid or liquid surfaces. However, in the present invention, the attraction of an opposite charge is used to deposit atomized particles, so that small atomized particles can also be aggregated and settled. The smaller the atomized particle size, the larger the specific surface area, and the atomized liquid per unit volume can be. The greater the charge capacity, the higher the efficiency of sterilization.

As an embodiment of the present invention, the liquid water atomization method is pressure spray, that is, liquid water such as pure water or brine is compressed to be sprayed out of the small holes to form an aerosol. The method is simple and convenient to operate, but the atomized particles formed are large in size, non-uniform, have a small charge capacity, and require pressurization equipment.

As an embodiment of the present invention, the liquid water atomization method is condensation after heating and evaporation, that is, a large amount of water vapor is introduced into the cooling chamber after the water is heated and boiled, and gaseous water molecules are cooled to gather into small liquid water droplets, so They are charged and sent to the surface of the tissue or object to be disinfected. The biggest advantage of this method is that the atomized particles are absolutely sterile without any microorganisms and pollutants; the disadvantages are serious energy consumption and low atomization efficiency.

As an embodiment of the present invention, the liquid water atomization method is a Venturi atomization method, which uses a Venturi tube to automatically suck liquid water such as purified water or saline in a high-speed air stream and breaks it into a mist. The atomizing particle size of this method is small, but it belongs to large equipment, with low aerosol concentration, high energy consumption, and especially serious noise.

As an embodiment of the present invention, the liquid water atomization method is mechanical agitation. Even if a high-speed rotating propeller blade is used, the liquid can be agitated into a droplet mist shape and blown to a destination through a fan and a duct. However, the atomization efficiency is low, the atomization particle size is large, uneven, and the charge capacity is small, and the disinfection and disinfection effect is poor. In addition, high energy consumption and noise are also its limitations.

In the present invention, the ultrasonic atomizing frequency range is 0.5 MHz to 5.0 MHz; using a battery or a rechargeable battery as a power source, in order to effectively control the repulsion or attraction between charged particles and tissues or objects, it is necessary to Increase the primary battery voltage. Make the potential difference between the output electrodes (that is, the voltage measured between the two electrodes when the external circuit is not connected) reach a certain high value. The commonly used output potential difference ranges between 300-100000V.

Under the condition that the power of the power source is not changed, the voltage will increase and the current intensity will decrease accordingly. This can reduce the discomfort of the human body when aerosol particles are discharged on the surface of the tissue. For this reason, the power of the power supply generally does not exceed 10w. The current intensity generally does not exceed 10 mA. But for the disinfection and sterilization of utensils or food surfaces. Power and current are not limited by the above. If indoor AC power (110 or 220V) is used, the AC needs to be rectified to DC first, and then boosted to the required voltage. Due to the large power of the indoor power supply, it is required to reduce the current intensity to a level that does not cause harm to the human body during the application and disinfection of human tissues.

Step S102: Bringing the Atomized Particles With Static Electricity of a First Polarity;

In specific implementation, the present invention can make the atomized particles electrostatically charged by directly or indirectly contacting the atomized particles with a high-voltage electrode. In this embodiment, the step of bringing the atomized particles with a first polarity of static electricity specifically includes: directly connecting the liquid water with a high-voltage output electrode having a first polarity, so that the atomized particles Take static electricity of the first polarity.

Experiments show that when tiny droplets (atomized particles) are separated from the liquid surface and lift off, they will carry some of the accumulated charges on the liquid surface, where the amount of charge carried is related to the voltage at which the liquid conducts, and it is related to the conductivity of the liquid droplets. Capacity (ie electrolyte concentration) is related to size, and to the surface tension of the liquid. However, under normal conditions, the atomized particles sent out by this method will be charged with the same polarity. If pure water with extremely high purity is used, a small amount of electrolyte (such as NaCl) can be added to reduce its resistivity and increase the charge capacity of the atomized particles.

In order to ensure the effect of disinfection and sterilization, the discharge deposition of charged physical and chemical particles on the surface of the object to be sterilized can generate enough active substances such as plasma and ozone. The electrostatic voltage between one electrostatic polarity and the second electrostatic polarity is not less than 300 volts.

As an embodiment of the present invention, the present invention may indirectly contact the atomized particles with a high-voltage electrode of at least 300 volts, that is, the step of charging the atomized particles with a first-polar static electricity specifically includes: The liquid water is sprayed into atomized particles with static electricity through a metal nozzle connected to a high voltage electrode under pressure, and the high voltage electrode has a first polarity. Since the liquid is electrostatically charged as it is squeezed through the nozzle, subsequent droplets can also be charged.

As an embodiment of the present invention, the step of bringing the atomized particles with a first-polar static electricity specifically includes: passing the atomized particles through at least one layer of a small-aperture conductive material mesh connected to a high-voltage electrode, so that the high-voltage electrode has a first polarity. That is, liquid water such as pure water or salt water is first atomized, and then the aerosol is passed through at least one layer of a small-aperture conductive material mesh (such as a metal mesh) connected to the high-voltage electrode, so that the atomized particles are Charged.

It should be noted that the present invention can use a high voltage generator to generate a DC high voltage output of not less than 300 volts. The so-called high voltage generator includes at least the following two types:

1) Use the oscillating boost component to convert the DC low voltage from the battery or other DC power source to AC high voltage; then use the rectifier component to rectify and convert the AC high voltage into pulsating DC high voltage. Due to the small size of the compact oscillating booster, it is difficult to radiate heat, and the internal insulation layer is liable to fail and cause a short circuit. It is best to cooperate with temperature monitoring and alarming measures.

2) The AC low voltage from the power grid is converted into DC high voltage by means of transformer and rectification. A transformer with primary and secondary coils is used to boost the low voltage first, and then a rectifier is used to rectify the AC high voltage to DC high voltage.

Step S103: the Object to be Disinfected is Charged With Static Electricity of a First Polarity;

In specific implementation, the electrostatic aerosol sterilization method of the present invention can be used for human body disinfection, and can also be used to disinfect appliances, food, or other conductive objects, that is, the objects to be disinfected are human bodies, appliances, and food.

When the object to be sterilized is a human body, due to the good electrical conductivity of the human body, only a hand (or other part) is connected to a high-voltage output electrode, so that the tissue of any part of the body can be charged with electrostatic charges. As an electrostatic conductor, when the human body is insulated from the ground (such as wearing shoes with soles of insulating material), a capacitance is formed between the body and the ground, which can store the charge conducted to the body, and its charging voltage varies with the amount of charge. Increase gradually. The stored charge is not evenly distributed in the human body, and is mainly concentrated on the skin surface. If the clothes are conductive, static electricity can also be carried on the clothes. When the charge on the surface of the skin is stored to a certain extent, when it meets the appropriate conditions (such as near the conductor, the surface has a tip, and the ambient humidity is high), it will discharge to generate sparks, but the time is very short.

That is, the step of electrostatically charging the object to be sterilized with the first polarity includes: keeping the human body insulated from the ground or other electrical conductors so that the human body directly or indirectly contacts the high-voltage electrode having the first polarity.

Specifically, in this embodiment, the human body is insulated from the ground after putting on shoes with rubber soles, so that a part of the body is connected by a wire or directly contacts an electrode, such as a finger with a conductive ring connected to the electrode or a hand holding the instrument The handle is connected to the electrode, so that the human body has static electricity of the first polarity.

As an embodiment of the present invention, the human body is insulated from the ground after wearing shoes with rubber soles, so that a part of the body is connected by a wire or directly contacts an electrode. The electrode does not output static electricity until the atomized particles reach the surface of the tissue. Only when the atomized particles reach the surface of the tissue to be sterilized, the electrode outputs static electricity of the opposite polarity to the atomized particles. In this way, part of the charged aerosol will settle on other parts of the body before reaching the tissue to be treated. Therefore, the sterilization efficiency is slightly worse than the previous charging method.

As an embodiment of the present invention, the human body is kept connected to the ground (bare feet or grounded with a wire), that is, the human body is at a zero potential, and the atomized particles are charged with a certain polarity of the charge, while the electrode of the other polarity is grounded. Once the charged particles approach the surface of the human body, they immediately discharge and settle. Therefore, this method is not suitable for disinfection and sterilization of deep tissues such as the nasopharyngeal cavity and the respiratory tract.

When the object to be sterilized is a non-human object, for example, for the sterilization and sterilization of utensils, food, or other conductive materials, it is usually necessary to use a lead to guide the high-voltage electrode with the first polarity to the conductive object or a container holding food. And keep the object to be disinfected insulated from the ground or other electrical conductors.

That is, the step of placing the object to be sterilized with static electricity of the first polarity includes: maintaining the object to be sterilized from the ground or other electrical conductors so that the human body directly or indirectly contacts the high-voltage electrode having the first polarity.

Step S104: Delivering the Atomized Particles With Static Electricity to a Predetermined Surface of the Object to be Disinfected;

In specific implementation, the present invention can use blowing, spraying or inhaling to deliver electrostatically atomized particles to a predetermined surface of an object to be sterilized, such as inhaling electrostatically atomized particles through a person's nasal cavity, or by pressure The sprayer sprays electrostatically atomized particles onto a predetermined surface of an object to be disinfected.

Step S105: Calculate the Time it Takes for the Atomized Particles to Reach a Predetermined Surface of the Object to be Disinfected;

In specific implementation, it is necessary to calculate and estimate the time required for the atomized particles to reach the predetermined surface of the object to be sterilized, so as to more accurately control the polarity of static electricity carried by the object to be sterilized and improve the effect of sterilization.

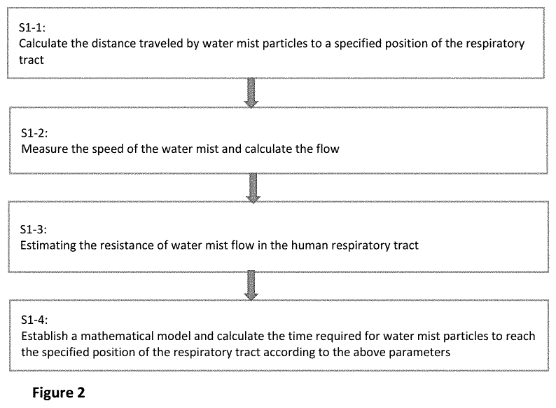

Referring to FIG. 2, the step S105 specifically includes:

Step S1-1: Calculate the distance required for the atomized particles to reach a predetermined surface of the object to be disinfected;

In specific implementation, firstly, it is necessary to estimate the distance that the atomized particles need to travel to reach the predetermined surface of the object to be disinfected (that is, the total stroke of the atomizing airflows). If the object to be disinfected is a non-human object, that is, food visible to the human eye , Appliances, etc., except for the special internal treatment of complex pipe fittings, the total travel of the atomizing airflow is generally easy to calculate or control at this time, or the above calculation of the settlement time is necessary.

If the object to be disinfected is a human body and its internal tissues, the calculation of the total stroke s of the atomized airflow is relatively complicated. Terminal bronchioles, respiratory bronchioles, alveolar ducts) and the length of the respiratory tract. A simple estimation method is to find out the relationship between gender, age, height and the length of the above parts based on the anatomy literature. For example, an adult male with a height of 170 cm has an average tracheal length (from the upper circular cartilage to the lower end connecting the left and right bronchial tubes) of 10.5 cm and a diameter of 2.0 cm. The right bronchus is 2.0-3.0 cm long and the left bronchi is 4.0-5.0 cm long. Adult women have slightly smaller trachea and bronchi, while children have shorter and thinner trachea. Then, the length of each airway part is estimated based on the user's gender, age and height.

Step S1-2: Measure the initial speed of the atomized particle airflow, and calculate the flow rate of the atomized particle airflow according to the initial speed;

In specific implementation, if the object to be sterilized is the human respiratory tract, that is, it is used for sterilization of the respiratory tract, the atomized particle airflow is related to the user's alveolar negative pressure and respiratory tract resistance. Even if the same person, the initial speed of each aerosol inhalation may be inconsistent, so a specially set flow velocity probe is needed to monitor the initial speed of the atomizing airflow at the nozzle to calculate the time when the atomized particles reach the action site.

According to the measured initial velocity Vₒ of the atomizing airflow and the aperture D at the nozzle or the outlet of the atomizing airflow, calculate the atomizing airflow rate I according to the following formula:

I=πD²Vₒ/4 (1)

Step S1-3: Estimating the resistance of the atomized particle airflow to a predetermined surface of the object to be disinfected;

In specific implementation, if the object to be disinfected is the human respiratory tract, first estimate the pressure difference P of the flow of atomized particle airflow: the power for air to enter the alveoli comes from the difference between the alveolar pressure Pₜ and the external air pressure. When inhaling calmly, the pressure difference is about 1-2 mmHg. During deep breathing, the pressure difference can increase to 5-20 mmHg or even higher. Ultrasound aerosol is mainly driven by its own breathing. Therefore, the driving pressure difference is the difference between the atmospheric pressure P_{c} and the alveolar pressure:

P=P_{c}−Pₜ (2)

It is necessary to estimate the airway ventilation resistance F: the airway ventilation resistance includes two aspects: elastic resistance Fₜ and inelastic resistance Fₑ. The former comes from the retraction of the lung tissue and the thoracic wall, and the latter comes from the frictional resistance of the airway airflow.

Step S1-4: A function model is established according to the flow rate, resistance and distance of the atomized particle airflow, and the time required for the atomized particle to reach a predetermined surface of the object to be disinfected is calculated through the function model.

In specific implementation, if the object to be disinfected is the human respiratory tract, the atomized particle gas flow is regarded as a viscous fluid, and the path through which it is ejected to a predetermined settling position is simplified as a pipe with a length of H and a diameter of h. The atomized airflow is calculated from the ejection time. The time t required to reach a specified location in the pipeline is the total stroke of the atomized airflow s, the airflow flow I, the pressure difference P between the two ends of the airflow, the airway resistance F, and the airflow viscosity coefficient η and the simulated pipeline geometry H and h:

t=f(s, I, P, F, η, H, h) (3)

Most aerosols are inhaled in a turbulent state. Through experimental tests, based on the consideration of the respiratory resistance F, an empirical formula is established to calculate the time t required for the atomized airflow to reach the predetermined deposition site. It is also possible to modify formula (3) by introducing the influence of Fe to make it approximate the conditions of turbulent airflow movement.

Step S106: Judging Whether the Atomized Particles Reach a Predetermined Surface of the Object to be Disinfected According to the Time, and if Yes, Proceed to Step S107; if Not, do Nothing.

In specific implementation, after the atomized particles reach a predetermined surface of the object to be disinfected, at this time, the atomized particles and the object to be disinfected have static electricity of the same polarity. Due to the repulsive force of the same-same charge, the atomization The particles are very stable and difficult to aggregate or settle to discharge.

Step S107: Change the Electrostatic Polarity of the Object to be Disinfected so that the Object to be Disinfected has Static Electricity of a Second Polarity, Where the Second Polarity is Opposite to the Polarity of the First Polarity.

In specific implementation, when the process that the atomized particles reach the predetermined surface of the object to be disinfected elapses for a predetermined time (that is, the time that the atomized particles reach the predetermined surface of the object to be disinfected calculated in step S105), that is, judgment The atomized particles have reached a predetermined surface of the object to be disinfected; at this time, the electrostatic polarity of the object to be disinfected is changed so that the object to be disinfected has static electricity of a second polarity, wherein the first The second polarity is opposite to the first polarity. In this way, due to the attractive effect of the opposite charge, the atomized particles immediately settle and settle on the predetermined surface of the object to be disinfected with bacteria and pathogenic microorganisms dormant and lose the charged charges. A large number of experiments have confirmed that the amount of liquid accumulated and charged on the surface of conductive objects (such as skin, metal plates, milk, and fruits) with a heterogeneous charge is an average of 2-3 higher than that of the same concentration without the electrical mist. Orders of magnitude. Theoretically speaking, the present invention uses electric field force to do work to overcome the surface energy between aerosol particles, so that they can fuse and aggregate and settle on the surface of a heterogeneously charged object. As long as the object continues to be charged, the charged mist particles will Continuously accumulate and settle on its surface, for example: due to the repulsion of the same sex, these charged particles cannot aggregate into large droplets, which not only maintains high stability but also does not collect and settle where it does not need to be settled, so it can go smoothly Enter any part of the body, such as the ears, mouth, nasopharynx, respiratory tract, lungs or any gaps, holes or complex pipes that need to be disinfected and sterilized, until the body or a charged object has static electricity of the opposite polarity, Only under the action of the electric field force, the surface discharges and settles.

The aerosol particles in the process of rapidly accumulating sedimentation of the heterogeneous charged tissue or object and lose the charged will produce a variety of mechanisms to inhibit and kill veruses:

(1) Pulsed Electric Shock

If the electrostatic potential between the charged mist particles and the charged object is sufficiently high (eg, not less than 1000V), the aerosol particles quickly fly to the surface of the charged object under the action of the electric field force, before approaching but not touching the surface of the object, Discharge will occur between the two. The aerosol particles that have lost their charge will impact the surface of the object under the action of the motion inertia, overcome the surface energy obstacle and settle smoothly. A large number of aerosol particles continue to fly towards the surface of a charged object, forming countless miniature lightnings, which will produce continuous electrical pulses against pathogenic microorganisms (mainly bacteria) lying on the surface of the object. This is similar to the high-voltage electric pulse sterilization principle used in the food industry in recent years. The discharge of aerosol particles can cause damage to pathogenic microorganisms, including permanent perforation of cell membranes.

(2) Plasma

A large number of miniature lightnings between charged mist particles and a charged object cause a large amount of miniature plasma to be generated in the area above the surface of the object. The plasma is composed of a large number of free electrons and ions, and contains a variety of chemically active particles that can be sterilized, mainly including reactive oxygen particles (ROS) and reactive nitrogen particles (RNS). ROS include O₂—, OH, O₃ (ozone) and H₂O₂, etc. RNS includes NO, NO₂, ONOO—, etc. Many of these active particles interact with living organisms in an aqueous environment. In the plasma sterilization process, ROS plays a very important role. When a small amount of O₂ is added to the ambient gas, its sterilization effect will be greatly improved. In the present invention, a large amount of chemically active particles generated by micro-plasma enter the liquid film on the surface of a tissue fire object with the agglomeration and sedimentation of aerosol particles, forming plasma-activated water, and performing pathogenic microorganisms attached to the surface of the tissue or object. Kill and suppress.

(3) Ozone Solution

A large amount of micro-plasma generated above the surface of a charged tissue or object contains many high-energy electrons, which collide with oxygen molecules in the air to promote the generation of oxygen ions, and the combination of oxygen molecules can form ozone molecules. Ozone molecules are easy to dissolve in water, so a certain percentage of ozone is melted in the water or saline liquid film deposited on the surface of the tissue capture object. The higher the electrostatic potential between the charged mist particles and the tissue or object, the higher the density of the aerosol particles, and the higher the ozone concentration in the surface liquid film.

Ozone has a strong oxidizing ability, and its redox potential is second only to F₂. The strong oxidizing properties of ozone can destroy bacteria's cell walls and ribonucleic acids, break down their macromolecular polymers such as DNA, RNA, proteins, lipids and polysaccharides, and penetrate into cell membrane tissues with water or physiological saline to cause permeability distortion. , Leading to lysis and death of cells, and the genetic genes, parasitic bacteria and other bacteria in the dead bacteria dissolved and denatured, to achieve the purpose of killing bacteria. The strong oxidizing power of ozone can also kill and inhibit virus reproduction. When the ozone concentration in the surface liquid film reaches more than 1 mg/L, it can kill harmful microorganisms such as spores, viruses, and fungi. Ozone can kill viruses and cysts that chlorine cannot kill, and it is thousands of times faster than chlorine. The ozone concentration in the liquid film is above 0.3 mg/L, which can kill all bacterial propagules on the surface within 1 minute, and effectively inhibit its reproduction.

(4) Brine Electrolysis

If salt water is used instead of clean water for charging after atomization, the micro-plasma generated by the discharge can activate salt water on the one hand and generate micro-electrolysis of salt water on the other. Micro-electrolysis produces chlorine gas, chloride ion, hypochlorite ion and highly oxidizing NaCIO ion. After these active substances are dissolved in the surface liquid film, the effect of sterilization and disinfection is enhanced. Studies have shown that the activated saline generated when the plasma discharge time reaches 120 s can reduce the survival efficiency of E. coli to 0.001%. Oxidizing particles such as H₂O₂ and O₃, which have a relatively stable life span in activated physiological saline, are still the main bactericidal substances.

(5) Salt Water Osmotic Pressure

If the concentration of saline used in the atomization is higher than the concentration of the pathogenic microbial cell fluid, due to the difference in osmotic pressure, the saline liquid film on the surface of the tissue or object will cause the microorganisms to die due to cell dehydration.

(6) Increase Humidity

Studies have found that when the ambient temperature is constant, when the air humidity reaches 50% or more, the cold virus will die quickly. So the dry conditions in winter just provide a comfortable environment for the virus. The technology of the invention can make a large number of water mist particles aggregate and settle on the mucosal surface of the nasopharyngeal cavity, increase the humidity of the nasopharyngeal cavity, and effectively inhibit the reproduction of rhinovirus that causes a cold. In addition, the increased humidity on the surface of the wound is also conducive to healing.

(7) Mechanical Flushing Effect

Pathogenic microorganisms and toxic substances on the surface of tissues or charged objects can be diluted by a large amount of liquid accumulated on the surface or taken away with water flow.

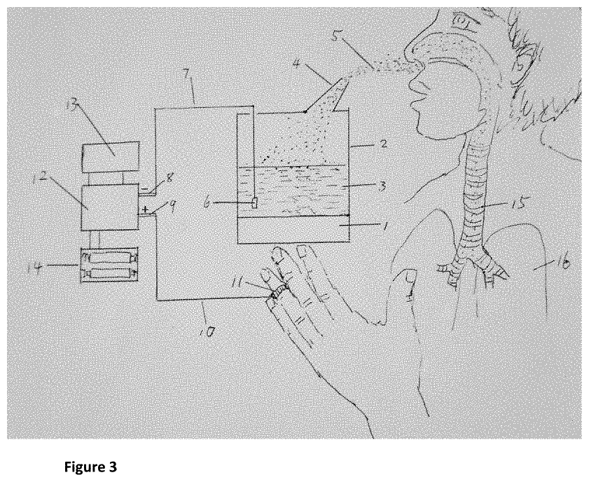

FIG. 3 is a schematic diagram of the method of the present invention for killing nasal viruses. Because the nasal cavity is at the forefront of the respiratory tract, the aerosol can reach the nasal cavity quickly, so the human body directly carries the static electricity opposite to the water mist particles, and there is no need to switch the polarity. In FIG. 3, 1 is an ultrasonic generator. 2 is a water container. 3 is sterile water or saline in the container. 4 is a water mist air outlet. 5 is an aerosol outlet, and the aerosol contains a large number of electrostatically charged water mist particles. 6 is a metal sheet. 7 is a wire connecting the metal sheet. 8 is the output negative pole of the high voltage electrostatic generator. 9 is the output positive pole of the high voltage electrostatic generator. 10 is a wire connecting the metal ring 11 and the positive electrode 9 of the electrostatic generator output. 12 is a high-voltage electrostatic generator. 13 is a system controller. 14 is the battery. 15 is the respiratory tract and 16 is the lungs.

The working principle of this nasal disinfection system is: the high voltage electrostatic generator 12 converts the 3V DC voltage of the power source 14 into a high voltage of not less than 300V. The high-voltage output negative electrode communicates with the sterile water or saline in the container 2 through the lead wire 7 and the metal sheet 6. The high-voltage output positive electrode communicates with the human body through the lead wire 10 and the metal ring 11, so that both the outer surface of the human body and the inner surface of the respiratory tract are positively charged. When the controller 13 issues an instruction, the ultrasonic generator 1 starts to work, so that the water in the container 2 is atomized. A large number of negatively charged water mist particles 5 are sprayed into the nasal cavity through the nozzle 4. These water mist particles successively discharge and settle on the inner surface of the positively charged nasal cavity, while generating a large amount of ozone and other active ions, thereby killing the virus in the nasal cavity.

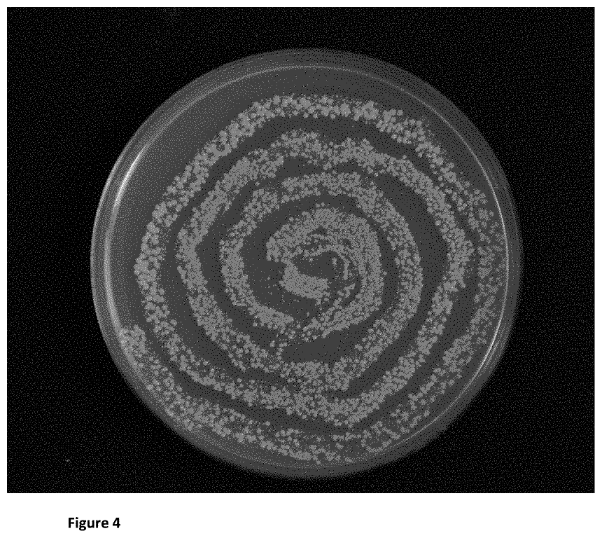

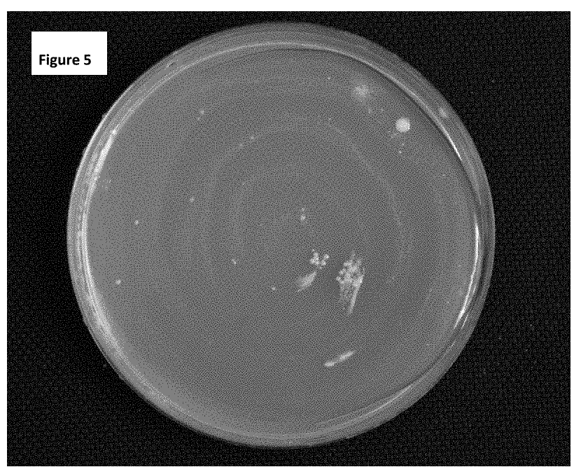

FIGS. 4 and 5 show the comparison of the number of colonies after using the system to spray ordinary water mist and charged water mist to volunteers' nasal cavity. FIG. 4 is to allow volunteers to inhale the aerosol generated with ordinary sterilized water for 30 seconds, and then wipe the sample with a sterile cotton swab in the nasal cavity. Then use the sampled cotton swab to draw a spiral on the agar surface of the petri dish, and then place the petri dish Incubate in a 36° C. incubator for 24 hours. Take out the photos. FIG. 5 follows the same procedure, but uses a charged aerosol. Comparing the number of colonies in a petri dish in the two cases (one small dot represents one bacterium), it can be seen that the sterilization effect of the method of the present invention is very effective, and the virus killing effect should also be effective.

Compared with the prior art, the electrostatic aerosol sterilization method, system and equipment of the present invention firstly atomize the liquid water atomized particles with the same polarity of static electricity as the object to be disinfected. When the particles reach the predetermined surface of the object to be sterilized, the object to be sterilized has static electricity of the opposite polarity. Due to the attractive effect of the opposite charge, the atomized particles quickly settle on the surface of the object and lose the charged charge. Under the multiple effects of plasma, ozone, chloride ion, salt ion, metal ion and pulse discharge generated in this process, the reproduction of pathogenic microorganisms on the object to be disinfected can be effectively suppressed or killed, and the object can be achieved. The surface sterilization greatly improves the effect of sterilization.

Moreover, the present invention improves the speed of aggregation and settlement of the atomized particles on the predetermined surface by calculating the time it takes for the atomized particles to reach the predetermined surface of the object to be disinfected, and switching the electrostatic polarity of the object to be disinfected according to the time control. The present invention is capable of treating and slowing down various respiratory diseases caused by viruses, including new pneumonia that threatens people all over the world, greatly improves the effect of disinfection and sterilization, is not only safe and reliable, but also has simple operation and low cost.

The technical features of the embodiments described above can be arbitrarily combined. In order to simplify the description, all possible combinations of the technical features in the above embodiments have not been described. However, as long as there is no contradiction in the combination of these technical features, It should be considered as the scope described in this specification.

The above-mentioned embodiments only express several implementation manners of the present invention, and their descriptions are more specific and detailed, but they cannot be understood as limiting the scope of the invention patent. It should be noted that, for those of ordinary skill in the art, without departing from the concept of the present invention, several modifications and improvements can be made, which all belong to the protection scope of the present invention. Therefore, the protection scope of the invention patent shall be subject to the appended claims.

1. An electrostatic aerosol disinfection and sterilization method, characterized in that the liquid water containing no medicine is atomized to obtain atomized particles of liquid water; and the atomized particles are charged with static electricity of a first polarity; Transporting the atomized particles with static electricity to a predetermined surface of the object to be disinfected, the object to be disinfected having static electricity of a first polarity; when the atomized particles reach the predetermined surface of the object to be disinfected, changing the electrostatic polarity of the object to be disinfected is such that the object to be disinfected has static electricity of a second polarity, wherein the second polarity is opposite to the polarity of the first polarity.

2. The electrostatic aerosol sterilization method according to claim 1, wherein the liquid water is sterilized in advance.

3. The electrostatic aerosol sterilization method according to claim 1, wherein the liquid water is salt water, in which the weight ratio of sodium chloride is not less than 0.1%

4. The electrostatic aerosol sterilization method according to claim 1, wherein the electrostatic voltage between the first electrostatic polarity and the second electrostatic polarity is not lower than 300 volts.

5. The electrostatic aerosol disinfection and sterilization method according to claim 1, wherein the step of atomizing the liquid water specifically comprises: atomizing the liquid water by a pressure spray method to obtain atomized particles.

6. The method for sterilizing and sterilizing an electrostatic aerosol according to claim 1, wherein the step of bringing the atomized particles with a static electricity of a first polarity specifically comprises:
directly connecting the liquid water The polarized high-voltage output electrode is connected, so that the atomized particles carry static electricity of a first polarity.

7. The electrostatic aerosol disinfection and sterilization method according to claim 1, wherein the step of atomizing liquid water containing no medicine to obtain atomized particles of liquid water specifically comprises: Sprayed under pressure through a metal nozzle connected to a high-voltage electrode, the high-voltage electrode has a first polarity.

8. The electrostatic aerosol disinfection and sterilization method according to claim 1, wherein the step of the object to be disinfected with static electricity of a first polarity comprises:
Keep the human body insulated from the ground or other electrical conductors, so that the human body directly or indirectly contacts the high-voltage electrode with the first polarity.

9. The electrostatic aerosol disinfection and sterilization method according to any one of claims 1 to 7, wherein the method further comprises:
Calculate the time that the atomized particles reach the predetermined surface of the object to be disinfected, and determine whether the atomized particles reach the predetermined surface of the object to be disinfected according to the time.

10. The electrostatic aerosol disinfection and sterilization method according to claim 8, wherein the step of calculating the time when the atomized particles reach a predetermined surface of the object to be disinfected specifically comprises:
Calculating the distance required for the atomized particles to reach a predetermined surface of the object to be disinfected;
Measuring the initial speed of the atomized particle airflow, and calculating the flow rate of the atomized particle airflow according to the initial speed;
Estimating the resistance of the atomized particle airflow to a predetermined surface process of the object to be disinfected;
A function model is established according to the flow, resistance and distance of the atomized particle airflow, and the time required for the atomized particles to reach a predetermined surface of the object to be disinfected is calculated through the function model.

11. An electrostatic aerosol disinfection and sterilization system, characterized in that the system includes:
Atomization module, for atomizing liquid water without any medicine to obtain atomized particles of liquid water;
An electrification module, configured to bring the atomized particles and the object to be disinfected with static electricity of a first polarity;
A conveying module for conveying electrostatically atomized particles to a predetermined surface of an object to be disinfected;
A polarity switching module, configured to change the electrostatic polarity of the object to be disinfected when the atomized particles reach a predetermined surface of the object to be disinfected, so that the object to be disinfected has static electricity of a second polarity, The second polarity is opposite to the first polarity.
